(19) United States
(12) Patent Application Publication
(10) Pub. No.: US 2024/0161910 A1
(43) Pub. Date: May 16, 2024

(54) EXAMINATION INFORMATION MANAGEMENT SYSTEM AND EXAMINATION INFORMATION MANAGEMENT DEVICE

(71) Applicant: Konica Minolta, Inc., Tokyo (JP)

(72) Inventors: Anna ONOBUCHI, Machida-shi, Tokyo (JP); Takanori ANDO, Hachioji-shi, Tokyo (JP)

(21) Appl. No.: 18/281,905

(22) PCT Filed: Feb. 24, 2022
(86) PCT No.: PCT/JP2022/007561

§ 371 Date: Sep. 13, 2023

(30) Foreign Application Priority Data
Mar. 17, 2021 (JP) 2021-044006

(51) Int. Cl. G16H 40/20; G16H 15/00

(52) U.S. Cl. CPC G16H 40/20; G16H 15/00

(57) ABSTRACT

The present invention provides an examination information management system capable of easily and appropriately setting an account level in a form suitable for an actual situation of a medical institution, even when a large number of related persons access each piece of examination information. The examination information management system includes: a transmission unit that transmits, to a user terminal used by a user, examination information regarding an examination performed in association with medical treatment and a control unit that controls transmission to the user terminal by the transmission unit, in accordance with a characteristic that is set for the user and is for indicating a range of the examination information accessible by the user. By control according to the characteristic, the control unit enables transmission of the examination information to the user terminal even when the user is a person other than a person in charge of the medical treatment.

### CROSS REFERENCE TO RELATED APPLICATIONS

This is the U.S. national stage of application No. PCT/JP2022/007561, filed on Feb. 24, 2022. Priority under 35 U.S.C. § 119(a) and 35 U.S.C. § 365(b) is claimed from Japanese Application No. 2021-044006, filed Mar. 17, 2021, the disclosure of which is also incorporated herein by reference.

### TECHNICAL FIELD

The present invention relates to an examination information management system and an examination information management device.

### BACKGROUND ART

A medical examination such as a genetic test involves not only a doctor in charge of medical treatment, but also a large number of related persons such as a person in charge of an examination in a hospital or outside, and a genetic counselor. Therefore, a large number of related persons access information (hereinafter, also simply referred to as “examination information”) regarding examination, such as an examination result and an examination intermediate report. Whereas, in a management system that stores and manages the examination information, it is necessary to set a predetermined access restriction in order to prevent leakage of the examination information that is personal information.

As a system that shares information regarding medical treatment, Patent Literature 1 discloses a medical image management server that can disclose medical information (medical image) with access restriction at a time of acquisition of a second opinion, acquisition of diagnosis in another institution, or the like. In the medical image management server described in Patent Literature 1, a reference level is set for each of various types of information (information regarding a patient, information regarding a facility, information regarding an examination, and information regarding treatment) included in medical information (medical image), whereas an account level is controlled for each account of a patient, an attending physician, and a diagnostician. Then, the reference level described above is compared with the account level described above to determine a disclosure level of the medical information escribed above. Specifically, the medical image management server described in Patent Literature 1 compares a reference level of certain medical information with an account level of a certain user (for example, a diagnostician belonging to an institution different from that of an attending physician), and the medical information is disclosed to the user (diagnostician) when these levels match.

### CITATION LIST

### Patent Literature

Patent Literature 1: JP 2014-044495 A

### SUMMARY OF INVENTION

### Technical Problem

The medical image management server described in Patent Literature 1 assigns a reference level to each piece of information included in the medical information and determines which information should be disclosed to which user. However, this method is very complicated, and in particular, assignment of a reference level to each piece of information may become a burden on a person who inputs data, and a possibility of occurrence of input errors or input omission increases as the number of pieces of data increases. Furthermore, when a large number of related persons access each piece of examination information, it is also necessary to adjust how and to which related person the account level should be assigned, and work becomes more complicated. However, in Patent Literature 1, the account level is controlled only for an account of a patient or a person in charge of medical treatment (an attending physician, a diagnostician). Therefore, for example, a person other than the person in charge of medical treatment, such as a doctor, a technician, or other experts in a laboratory cannot access medical information.

The present invention has been made on the basis of the findings described above, and in particular, an object thereof is to provide an examination information management system and an examination information management device capable of easily and appropriately setting an account level in a form suitable for an actual situation of a medical institution, even when a large number of related persons access each piece of examination information.

### Solution to Problem

The problems described above are solved by an examination information management system including: a transmission unit that transmits, to a user terminal used by a user, examination information regarding an examination performed in association with medical treatment; and a control unit that controls transmission to the user terminal by the transmission unit, in accordance with a characteristic that is set for the user and is for indicating a range of the examination information accessible by the user, in which, by control according to the characteristic, the control unit enables transmission of the examination information to the user terminal even when the user is a person other than a person in charge of the medical treatment.

Furthermore, the problems described above are solved by an examination information management device for managing transmission of examination information regarding an examination performed in association with medical treatment, the examination information management device including: a control unit that controls transmission of the examination information such that the examination information regarding the medical treatment can be transmitted to a user terminal used by the user even when the user is a person other than a person in charge of the medical treatment, in accordance with a characteristic for indicating a range of the examination information accessible by the user.

### Advantageous Effects of Invention

According to the present invention, there are provided an examination information management system and an examination information management device capable of easily and appropriately setting an account level in a form suitable for an actual situation of a medical institution, even when a large number of related persons access each piece of examination information.

### BRIEF DESCRIPTION OF DRAWINGS

FIG. 1 is a configuration diagram illustrating an example of a configuration of an examination information management system according to an embodiment of the present invention.

FIG. 2 is a block diagram illustrating an example of a configuration of a terminal device.

FIG. 3 is a block diagram illustrating an example of a configuration of a server device.

FIG. 4 is a list of examination information displayed on a display unit of a terminal device used by a person in charge of medical treatment in a certain setting.

FIG. 5 is an example of information displayed on a display unit of a terminal device used by another person in charge.

FIG. 6A is an example of information displayed on the display unit of the terminal device when access authority is changed to “all in the same clinical department”, and FIG. 6B is an example of information displayed on the display unit of the terminal device when access authority is changed to “all in the same medical institution”.

FIG. 7 is an example of information displayed on the display unit of the terminal device used by another person in charge when “all in the same clinical department”, which is access authority different from that of the person in charge of medical treatment, is selected for a counselor in charge.

FIG. 8 is an example of a screen for selecting examination information to be downloaded.

FIG. 9 is a list of examination information displayed on a display unit of a terminal device used by a manager.

FIG. 10 is an example of information displayed on the display unit of the terminal device used by the manager.

FIG. 11 is an example of information displayed on the display unit of the terminal device used by the manager.

### DESCRIPTION OF EMBODIMENTS

Hereinafter, an embodiment of the present invention will be described in detail with reference to the drawings. Note that the present invention is not limited to the illustrated forms.

[Examination Information Management System]

An embodiment of the present invention relates to an examination information management system in which examination information regarding an examination performed in association with medical treatment is input and stored. Further, the examination information management system described above has an access restriction function for restricting users who can access each piece of examination information.

Note that examples of the examination information described above include, but are not limited to, information related to a genetic test, image diagnosis such as X-ray and CT, blood examination, pathological diagnosis, and the like.

In addition, “access” in this specification means reading or downloading information stored in a storage unit of a server device to a terminal device (user terminal) used by each user, and editing (adding, changing, deleting, and the like) information stored in the storage unit of the server device from the terminal device used by each user.

[Configuration]

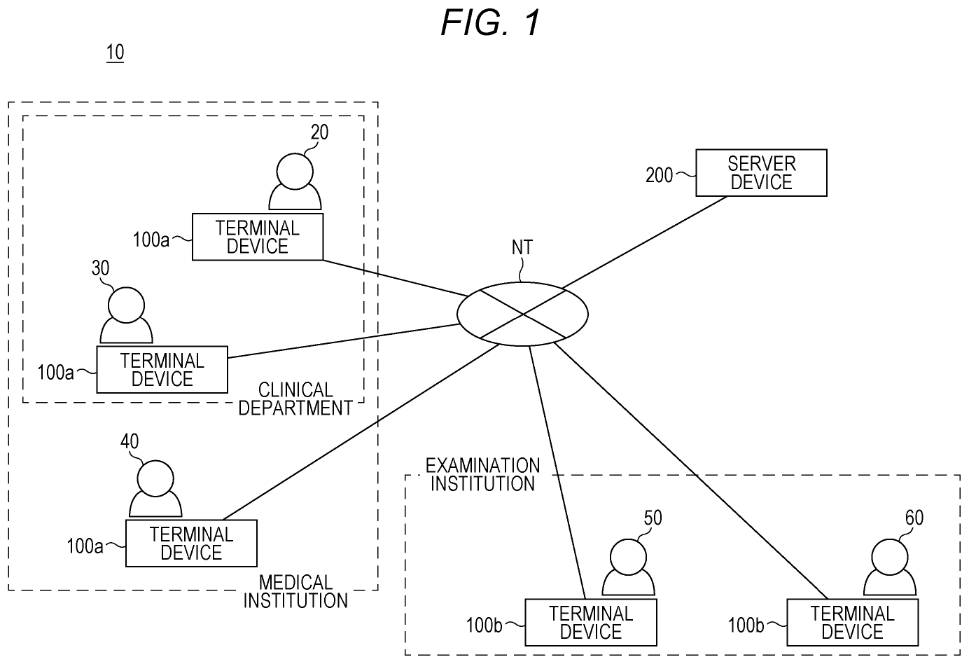

FIG. 1 is a configuration diagram illustrating an example of a configuration of an examination information management system according to an embodiment of the present invention. As illustrated in FIG. 1, an examination information management system 10 includes a plurality of terminal devices 100 and a server device 200. Each of the plurality of terminal devices 100 is connected to the server device 200 via a network NT. The network NT is, for example, a public network such as the Internet or a local network such as an intranet.

The terminal device 100 (terminal device 100a) is a terminal used by a user belonging to a medical institution to access medical information. The user described above includes a person in charge of medical treatment, that is, a person in charge of consultation and treatment of a patient (hereinafter, also simply referred to as a “person in charge of medical treatment”). Examples of the person in charge of medical treatment include an attending physician of the patient and a doctor in charge of medical treatment. In addition, the user described above includes a person other than the person in charge of medical treatment. The person other than the person in charge of medical treatment means a person who is not directly involved in consultation and treatment of the patient, that is, includes a person who is not the person in charge of medical treatment but is involved in medical treatment. For example, the person other than the person in charge of medical treatment may include: a doctor or technician in a laboratory who manages an examination (hereinafter, also simply referred to as “examination manager”); a healthcare provider who assists medical treatment; and an expert (hereinafter, also simply referred to as a “counselor in charge”) who provides specialized knowledge about examination results, such as a counselor.

Furthermore, the terminal device 100 (terminal device 100b) may be a terminal used by a user belonging to an examination institution to transmit medical information and other information to the server device 200 (and other users via the server device 200). At this time, the user described above may include: a technician in charge of a plurality of examinations in an examination institution (or an examination department of a hospital) (hereinafter, also simply referred to as a “person in charge of an examination”); and a technician authorized to manage a plurality of examinations at an examination institution (or an examination department of a hospital) (hereinafter, also simply referred to as an “authorized manager”).

Note that the terminal device 100a and the terminal device 100b described above are only discriminations based on a role of the user (whether the user belongs to a medical institution or an examination institution). As will be described later, information may be transmitted from the terminal device 100a of the user belonging to the medical institution to the server device 200, or the terminal device 100b of the user belonging to the examination institution may receive information from the server device 200.

Note that individual users may use the same terminal device or may use different terminal devices. In this specification, even in a case where the individual users use the same terminal device, when each user logs in with a different account to use the terminal device, the terminal device is regarded as a separate terminal device 100a or 100b for each user.

FIG. 1 illustrates the terminal device 100a used by a person in charge 20 of medical treatment; the terminal device 100a used by a counselor in charge 30; the terminal device 100a used by another person in charge 40 who is also a superior of the person in charge 20 of medical treatment; the terminal device 100b used by a person in charge 50 of an examination; and the terminal device 100b used by an authorized manager 60. In the present embodiment, an example will be exemplified in which the person in charge 20 of medical treatment and the counselor in charge 30 belong to the same clinical department (Dept. of Oncology) in the same medical institution (AAA Hospital), the other person in charge 40 belongs to a different department of the same medical institution (AAA Hospital) as the person in charge 20 of medical treatment and the counselor in charge 30, and the person in charge 50 of the examination and the authorized manager 60 belong to an external examination institution.

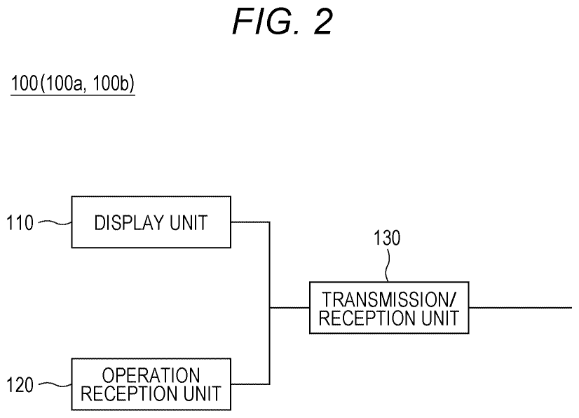

FIG. 2 is a block diagram illustrating an example of a configuration of the terminal device 100a and the terminal device 100b. Each of the terminal device 100a and the terminal device 100b includes a display unit 110, an operation reception unit 120, and a transmission/reception unit 130. The terminal device 100 is, for example, a personal computer (PC). The display unit 110 is a portion that performs display, and is, for example, a liquid crystal display, an organic electro luminescence (EL) display, a cathode ray tube (CRT) display, or the like. The operation reception unit 120 is a portion that receives a user's operation, and is, for example, a keyboard, a mouse, a trackball, a touch pad, a touch panel, or the like. In a case where the operation reception unit 120 is a touch panel, the display unit 110 and the operation reception unit 120 may be disposed so as to overlap with each other. The transmission/reception unit 130 is a communication device for transmitting and receiving information to and from the server device 200 via the network NT. In this specification, the transmission/reception unit 130 may be referred to as a “transmission unit” when transmitting information and may be referred to as a “reception unit” when receiving information.

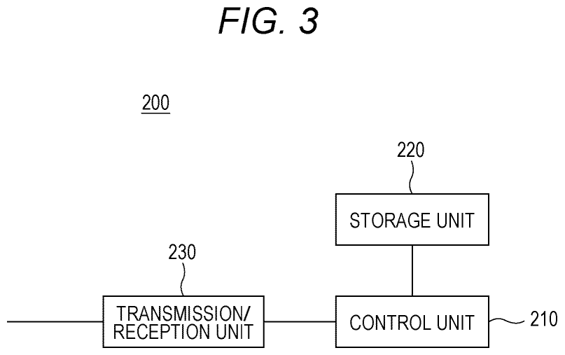

FIG. 3 is a block diagram illustrating an example of a configuration of the server device 200. The server device 200 includes a control unit 210, a storage unit 220, and a transmission/reception unit 230. The server device 200 is, for example, a cloud server. Therefore, the control unit 210, the storage unit 220, and the transmission/reception unit 230 included in the server device 200 are not necessarily provided in a physical sense, and it is sufficient that functions for realizing operations of these components are constructed on the cloud. Alternatively, the server device 200 may be a server device (in-house server or the like) installed in a local network.

The control unit 210 is, for example, a control device such as a central processing unit (CPU). The control unit 210 executes a predetermined application on the basis of an instruction from the terminal device 100 and controls a screen to be displayed on the display unit 110 of the plurality of terminal devices 100. The storage unit 220 includes, for example, a cache memory, a main storage device (a random access memory (RAM) or the like), and an auxiliary storage device (a hard disk drive (HDD), a solid state drive (SSD), or the like). The transmission/reception unit 230 is a communication device for transmitting and receiving information to and from each of the terminal device 100a and the terminal device 100b via the network NT. In this specification, the transmission/reception unit 230 may be referred to as a “transmission unit” when transmitting information and may be referred to as a “reception unit” when receiving information.

[Operation Example]

(Access Authority)

The server device 200 stores, in the storage unit 220, examination information about an examination performed by the person in charge 50 of the examination. Then, the examination information described above is transmitted from the transmission/reception unit 230 to the terminal device 100a used by the person in charge 20 of medical treatment. Note that, in this specification, a terminal device used by a certain user means a terminal device that the user has logged in with the own account.

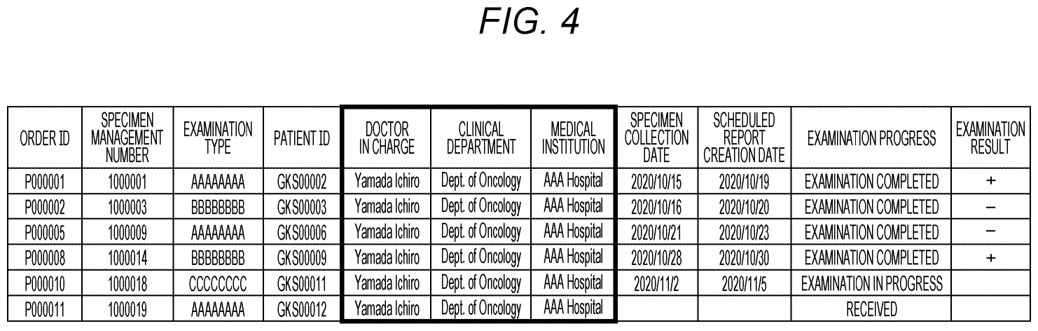

FIG. 4 is a list of examination information displayed on the display unit 110 of the terminal device 100a used by the person in charge 20 of medical treatment (Dr. Yamada Ichiro) in a certain setting. The terminal device 100a receives examination information transmitted from the transmission/reception unit 230 of the server device 200 by the transmission/reception unit 130 and displays the examination information on the display unit 110. The displayed examination information includes: “order ID” which is a request number when a medical institution (AAA Hospital) to which the person in charge 20 of medical treatment belongs requests an examination to an examination institution to which the person in charge 50 of the examination belongs; “specimen management number” which is a unique number granted by the examination information management system 10 and indicating the examination; “examination type” indicating a type of the examination, “patient ID” which is a number assigned by the medical institution (AAA Hospital) and indicating a patient; “doctor in charge” which is a number indicating a doctor in charge; “clinical department” which is a number indicating a clinical department to which the doctor in charge belongs; “medical institution” indicating a medical institution to which an attending physician belongs; “specimen collection date” indicating a collection date of a specimen to be subjected to the examination; “scheduled report creation date” which is a scheduled creation date of an examination report; and “examination progress” indicating a progress status of each examination.

In addition, as illustrated in FIG. 4, an examination result may be simply displayed on the display unit 110 of the terminal device 100a. In FIG. 4, in “examination result”, “+” is displayed when a specimen examination result is positive, and “−” is displayed when the specimen examination result is negative. As a result, the person in charge 20 of medical treatment can preferentially download an examination report regarding the examination whose “examination result” is “+” and check contents in detail, and thus, work efficiency of the person in charge 20 of medical treatment is improved.

Note that the contents of the examination information transmitted and received and displayed on the display unit 110 of the terminal device 100a are not limited to those described above, and may be only a part of those described above, or may further include, for example, information such as whether or not an insurance is applied, a date of receipt of the specimen by the examination institution, a scheduled completion date of the examination, and a name of the examination institution.

FIG. 4 illustrates examination information displayed on the display unit 110 of the terminal device 100a used by the person in charge 20 of medical treatment (Dr. Yamada Ichiro) when the person in charge 20 of medical treatment is granted with access authority for only examination information related to an examination of which the person in charge 20 is in charge.

In the present embodiment, the authorized manager 60 who is also a responsible person in the examination institution sets a characteristic for indicating a range of examination information accessible by each person in charge 20 of medical treatment, that is, which examination information can be accessed. However, the authorized manager may be a responsible person in the medical institution. Then, in accordance with the set characteristic, examination information for which the access authority is granted is displayed on the display unit 110 of the terminal device 100a used by each person in charge 20 of medical treatment. Note that the set characteristic is stored in the storage unit 220, and the control unit 210 can refer to the characteristic stored in the storage unit 220 as appropriate.

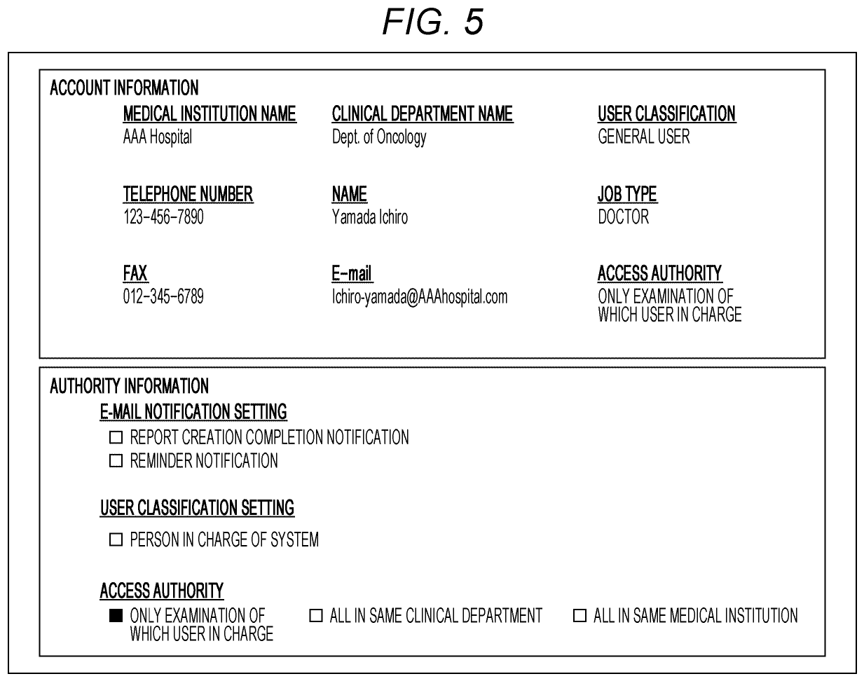

FIG. 5 is an example of information displayed on the display unit 110 of the terminal device 100b used by the authorized manager 60.

On the display unit 110 of the terminal device 100b used by the authorized manager 60 who is a responsible person in the examination institution, account information of each person in charge 20 of medical treatment is displayed. Then, the authorized manager 60 can set a range of examination information accessible by each person in charge 20 of medical treatment from the screen.

The account information includes information such as: “medical institution name” indicating a medical institution to which each user belongs; “clinical department name” indicating a clinical department to which the user belongs; “user classification” indicating whether or not the user is a general user who can only receive examination information or a person in charge of the system who can grant access authority; contact information such as “telephone number”, “FAX number”, and “e-mail address”; name; “job type” such as a doctor, a healthcare provider, and a genetic counselor; and “range of access authority”.

In FIG. 5, to Dr. Yamada Ichiro, who is the person in charge 20 of medical treatment, access authority for only an examination of which Dr. Yamada Ichiro is in charge is granted. At this time, the control unit of the server device 200 permits transmission of only examination information whose medical institution name (AAA Hospital), clinical department name (Dept. of Oncology), and name (Yamada Ichiro) match, to the terminal device 100a used by Dr. Yamada Ichiro from the transmission/reception unit 230.

In FIG. 5, when the authorized manager 60 changes the access authority (that is, the characteristic indicating the range of accessible examination information) in the authority information from “only examination of which the user in charge” to “all in the same clinical department”, Dr. Yamada Ichiro is granted with access authority for all examinations in the clinical department to which Dr. Yamada Ichiro belongs. Furthermore, when the authorized manager 60 changes the access authority in the authority information to “all in the same medical institution”, access authority for all examinations of the medical institution to which Dr. Yamada Ichiro belongs is granted to Dr. Yamada Ichiro.

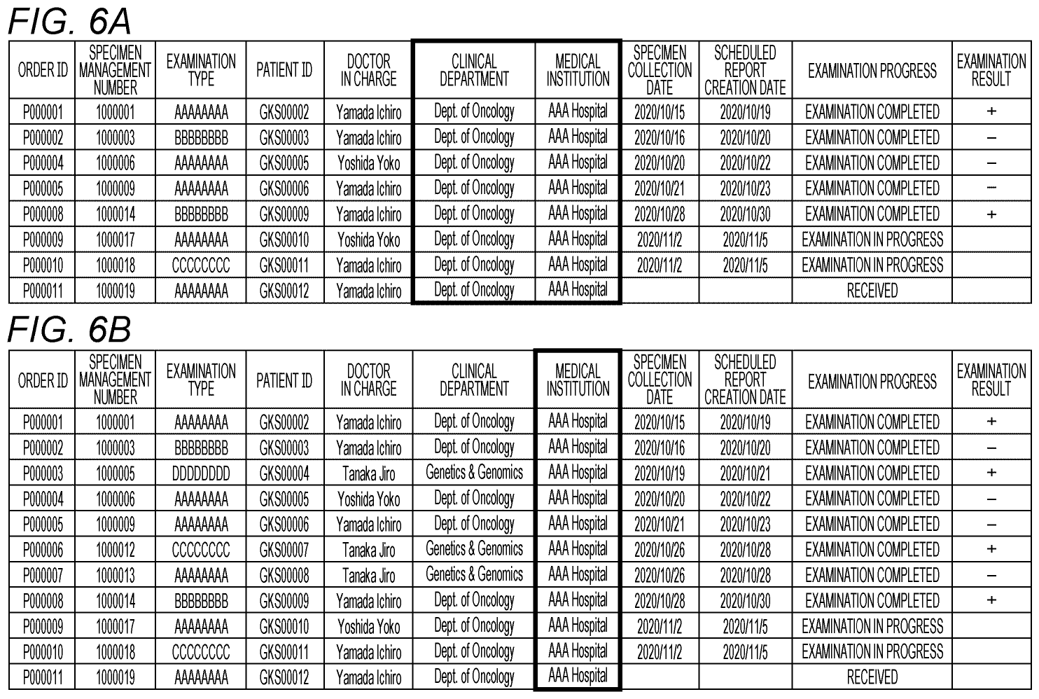

FIG. 6A is an example of information displayed on the display unit 110 of the terminal device 100a used by Dr. Yamada Ichiro, when the access authority is changed to “all in the same clinical department”. At this time, the control unit of the server device 200 permits transmission of only examination information whose medical institution name (AAA Hospital) and clinical department name (Dept. of Oncology) match the account information of Dr. Yamada Ichiro, to the terminal device 100a used by Dr. Yamada Ichiro from the transmission/reception unit 230. Then, the display unit 110 also displays other examination information (examination information in which a doctor in charge is Dr. Yoshida Yoko) whose medical institution is AAA Hospital and clinical department is Dept. of Oncology.

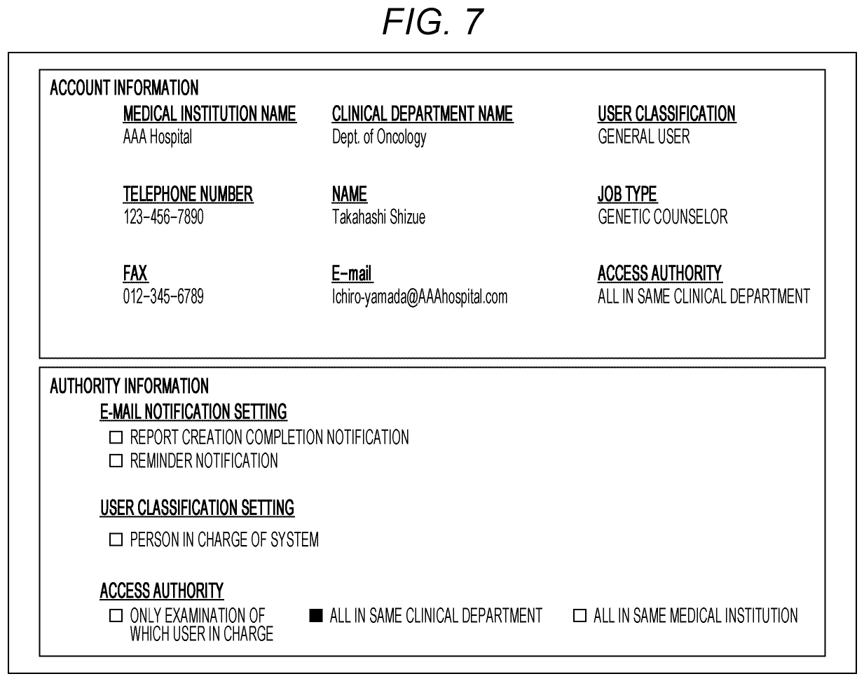

FIG. 6B is an example of information displayed on the display unit 110 of the terminal device 100a used by Dr. Yamada Ichiro, when the access authority is changed to “all in the same medical institution”. At this time, the control unit of the server device 200 permits transmission of only examination information whose medical institution name (AAA Hospital) matches the account information of Dr. Yamada Ichiro, to the terminal device 100a used by Dr. Yamada Ichiro from the transmission/reception unit 230. Then, the display unit 110 also displays other examination information (examination information in which a doctor in charge is Yoshida Yoko or Tanaka Jiro) whose medical institution is AAA Hospital.

Whereas, the authorized manager 60 can refer to account information of the counselor in charge 30 and can give access authority different from the access authority of the person in charge 20 of medical treatment, to the counselor in charge 30. FIG. 7 illustrates a state in which “all in the same clinical department”, which is access authority different from that of Dr. Yamada Ichiro, has been selected for Dr. Takahashi Shizue, who is the counselor in charge 30. In this way, the authorized manager 60 can refer to account information of a plurality of users (the person in charge 20 of medical treatment or other healthcare providers, genetic counselors, and the like) and independently set the range of the examination information accessible by these users.

For example, although not involved in medical treatment, a healthcare provider or a genetic counselor may be in charge of a large number of examinations requested from the same clinical department. In addition, a genetic counselor may be in charge of requesting a genetic test to the outside and receiving a result regarding a certain clinical department at once. At this time, if it is attempted to determine to which healthcare provider or genetic counselor the access authority is to be granted for each examination, setting work for the access authority becomes very complicated, and thus input errors are likely to occur. Furthermore, it is very time-consuming to access examination information via the person in charge of medical treatment every time when it is desired to refer to the examination information. On the other hand, it is possible to permit a necessary user to access necessary information by an easier method, by giving access authority for all pieces of the examination information in the same clinical department to a healthcare provider or a genetic counselor not involved in medical treatment. In addition, by limiting the accessible examination information to the same clinical department, it is possible to suppress occurrence of a situation in which a larger number of people than necessary can access the examination information.

In addition, depending on the medical institution, a chief clinical technologist of a laboratory may collectively manage examination information of a plurality of clinical departments, and request genetic tests to the outside, receive results, and register the results in medical records. Even in such a case, if the access authority is to be granted to the chief clinical technologist for each examination, work becomes very complicated. On the other hand, if access authority for all pieces of the examination information in the medical institution is granted to the chief clinical technologist, a necessary user can be permitted to access the necessary information by an easier method.

Alternatively, in a medical institution in which a person in charge of medical treatment manages a genetic test for medical treatment of which the person is in charge, the person in charge 20 of medical treatment may be granted with access authority only for the examination of which the person is in charge.

In this way, by setting the range of accessible examination information for each user, it is possible to more easily grant the access authority in accordance with an actual situation of the medical institution. In addition, by setting the range of accessible examination information in accordance with an attribute related to a belonging destination of the user, it is possible to more easily grant the user with access authority for necessary examination information. For example, since the range of the examination information accessible by the user is set for each user, even in a case where the user is a person other than a person in charge of medical treatment, it is possible to access examination information regarding the medical treatment.

Note that, by selecting a person in charge of the system in the “authority information” of FIG. 5, it is also possible to grant the user (Dr. Yamada Ichiro) with authority to set access authority of the user or another user.

(Download of Examination Information)

Each of the users (the person in charge 20 of medical treatment and the counselor in charge 30) logs in the examination information management system 10 with the own account from the terminal device 100a. Then, among pieces of examination information stored in the storage unit 220 of the server device 200, examination information accessible by the user can be downloaded to the terminal device 100a used by the user.

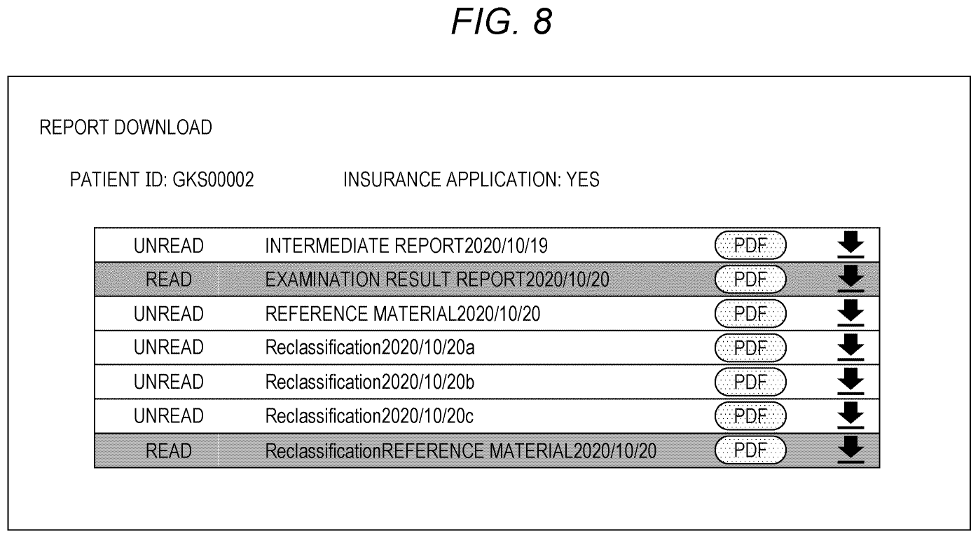

FIG. 8 is an example of a screen for selecting examination information to be downloaded.

The user may download all pieces of the downloadable examination information at once or may select and download specific examination information such as for each patient, for each doctor in charge, for each examination type, and examination information after a predetermined date and time. FIG. 8 indicates information displayed on the display unit 110 when the user inputs to the operation reception unit 120 of the terminal device 100a so as to display a list of examination information that can be downloaded for an examination performed on a specimen collected from a certain patient (patient ID: GKS00002).

When the transmission/reception unit 130 of the server device 200 receives a request for the list of the examination information about the patient described above transmitted from the transmission/reception unit 230 of the terminal device 100a, the control unit 210 of the server device 200 permits transmission of the list extracted from the examination information stored in the storage unit 220, from the transmission/reception unit 230 of the server device 200 to the transmission/reception unit 130 of the terminal device 100a.

Note that the storage unit 220 stores information about which examination information the user has already downloaded, together with account information of each user. Then, the control unit 210 permits transmission of, together with the list described above, information about whether or not each piece of examination information included in the list has been downloaded to the terminal device 100a of the user, to the transmission/reception unit 130 of the terminal device 100a.

The terminal device 100a having received the various types of information described above by the transmission/reception unit 130 displays a type of the examination information, whether or not the examination information has been downloaded, and a file type (PDF, Word file, Excel file, and the like), on the display unit 110. The type of the examination information includes an intermediate report, a final report (examination result report), and other related materials. In FIG. 8, whether or not the examination information has already been downloaded is displayed as “unread” or “read” together with the type of the examination information. Display of a color tone or the like may be changed between the unread examination information and the read examination information so that it is easy to discriminate between unread or read. Then, when the user selects information desired to be downloaded, the selected information is transmitted from the transmission/reception unit 230 of the server device 200 to the transmission/reception unit 130 of the terminal device 100a used by the user, and the terminal device 100 displays the received examination information on the display unit 110.

Note that, when the user logs in, the control unit 210 of the server device 200 may cause the transmission/reception unit 230 of the server device 200 to transmit information (notification) indicating that new examination information has been registered, to the terminal device 100 used by the user. Alternatively, when the new examination information is stored in the storage unit 220, for the user related to the examination information, the control unit 210 of the server device 200 may transmit a notification indicating that the new examination information has been registered, to an e-mail address or a telephone number registered for the user.

Then, after the notification or the e-mail is transmitted, when the user does not download the examination information by the procedure described above and the server device does not transmit the examination information until a predetermined period elapses, the control unit 210 of the server device 200 may cause the transmission/reception unit 230 to transmit reminder information to the user. The reminder information may be set to be displayed on the display unit 110 of the terminal device 100a as a notification at the time of login, or the notification may be transmitted to an e-mail address, or a telephone number registered for the user. In particular, by reminding an examination result report, it is possible to more reliably prevent overlooking of the examination result.

Whether or not to transmit the information (notification) indicating that the new examination information described above is registered and the reminder information can be set for each user (see FIG. 5 and FIG. 7).

(From Examination Request to Upload of Examination Information)

When a person in charge such as a doctor in charge of medical treatment, a genetic counselor, and a chief clinical technologist requests a medical examination such as a genetic test to an external examination institution after an interview with a patient, a record of the examination is created in the examination information management system 10. Thereafter, a specimen prepared by, for example, collecting blood or collecting tumor tissue, cells, or the like at a medical institution is collected by a collection trader and delivered to the examination institution.

When information indicating a collection date of the specimen described above and input from the operation reception unit 120 of the terminal device 100b used by a user belonging to the examination institution is received by the transmission/reception unit 230 of the server device 200 and registered in the examination information management system 10, the examination information management system 10 calculates and registers a scheduled completion date of an examination report. This scheduled completion date may also be transmitted to the terminal device 100a used by the user belonging to the medical institution and displayed on the display unit 110 as part of the examination information, under the control of the control unit 210 (see FIGS. 4, FIG. 6A and FIG. 6B).

When the collection date of the specimen is registered, the examination information management system 10 calculates the scheduled completion date of the examination report on the basis of business days excluding holidays such as Saturday and Sunday and public holidays. In the examples illustrated in FIGS. 4, FIG. 6A, and FIG. 6B, after the lapse of two business days excluding Saturday and Sunday and Culture Day (November 3) with respect to the specimen collection date is set as the scheduled completion date of the examination report. Note that the examination information management system 10 may calculate the scheduled completion date of the examination report, excluding non-business days for each examination institution.

In addition, the examination information management system 10 may register a date on which the specimen has arrived at the examination institution (a reception date of the specimen) and the scheduled completion date of the examination calculated from the reception date of the specimen.

Further, the examination information management system 10 may store, in the storage unit 220, examination progress information indicating being before collection of the specimen (receiving), examination being in progress by the examination institution, being after end of the examination and completion of the examination report (examination completed), and being after cancellation, and cause the examination progress information to be transmitted to the terminal device 100a used by the user belonging to the medical institution and displayed on the display unit 110, under the control of the control unit 210 (see FIGS. 4, FIG. 6A and FIG. 6B).

When the examination is ended and creation of the examination report is completed, the person in charge 50 of the examination belonging to the examination institution transmits the created examination report from the own terminal device 100b to the server device 200. The server device 200 stores the examination report received by the transmission/reception unit 230 into the storage unit 220.

When the examination report is registered, under the control of the control unit 210, the examination information management system 10 may transmit information (notification) indicating that new examination information has been registered, from the transmission/reception unit 230 to the transmission/reception unit 130 of the terminal device 100a used by the user belonging to the medical institution, or from the transmission/reception unit 230 to an e-mail address or a telephone number of the user belonging to the medical institution. Alternatively, regarding the registered examination report, the control unit 210 may permit transmission of information (notification) indicating that the new examination information described above has been registered after approval by the authorized manager 60 is granted, as a state of waiting for approval by the authorized manager 60. Further, under the control of the control unit 210, the examination information management system 10 may transmit an examination progress status and a notification indicating update of the examination information, from the transmission/reception unit 230 to the transmission/reception unit 130 of the terminal device 100a used by the user belonging to the medical institution, or from the transmission/reception unit 230 to an e-mail address or a telephone number of the user belonging to the medical institution.

(Authorized Manager 60)

The authorized manager 60 belongs to the examination institution, and checks and approves contents of the examination report uploaded by the person in charge 50 of the examination.

In the examination information management system 10, the presence or absence of manager authority is set in the account of the user. Then, manager authority is granted to the account of the authorized manager 60. Note that the manager authority is set so as to be able to be granted and canceled.

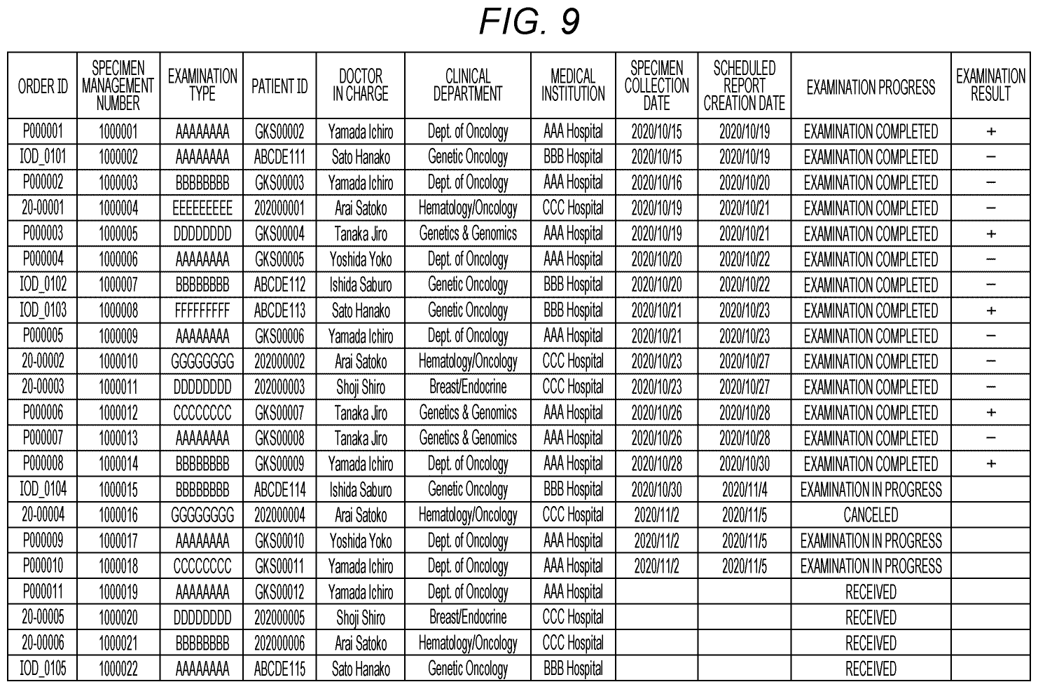

In order to enable the management and approval described above, the control unit 210 of the server device 200 permits transmission of all pieces of the examination information to the terminal device 100b used by the user having the manager authority (the authorized manager 60). FIG. 9 is a list of examination information displayed on the display unit 110 of the terminal device 100b used by the authorized manager 60. At this time, the control unit of the server device 200 permits transmission of all pieces of the examination information to the terminal device 100b used by the authorized manager 60 from the transmission/reception unit 230, regardless of whether matching or mismatching the account information. Then, all pieces of the examination information is displayed on the display unit 110.

When the authorized manager 60 checks and approves the examination report, the control unit 210 of the server device 200 permits transmission of examination information including the examination report to be checked and approved, from the transmission/reception unit 230 to the terminal device 100b used by the authorized manager 60. The authorized manager 60 displays the examination report received by the transmission/reception unit 130 of the terminal device 100b on the display unit 110 to check the contents. Further, from the operation reception unit 120 to the terminal device 100b, the authorized manager 60 inputs approval when the contents are appropriate, and inputs returning to the person in charge 50 of the examination together with a comment when the contents are incomplete. The terminal device 100b transmits the input information described above from the transmission/reception unit 130 to the server device 200. When the transmission/reception unit 230 receives information that approves a certain examination report, the server device 200 transmits information (notification) indicating that the examination report has been registered as new examination information, to another user (the person in charge 20 of medical treatment, the counselor in charge 30, or the like). When the transmission/reception unit 230 receives information indicating that the examination report is returned, the server device 200 transmits information (notification) indicating that the examination report is returned to the person in charge 50 of the examination together with a comment.

In this way, quality of the examination report is ensured by permitting transmission to the user in the medical institution (the person in charge 20 of medical treatment, the counselor in charge 30, and the like) after the authorized manager 60 checks and approves the contents of the examination report.

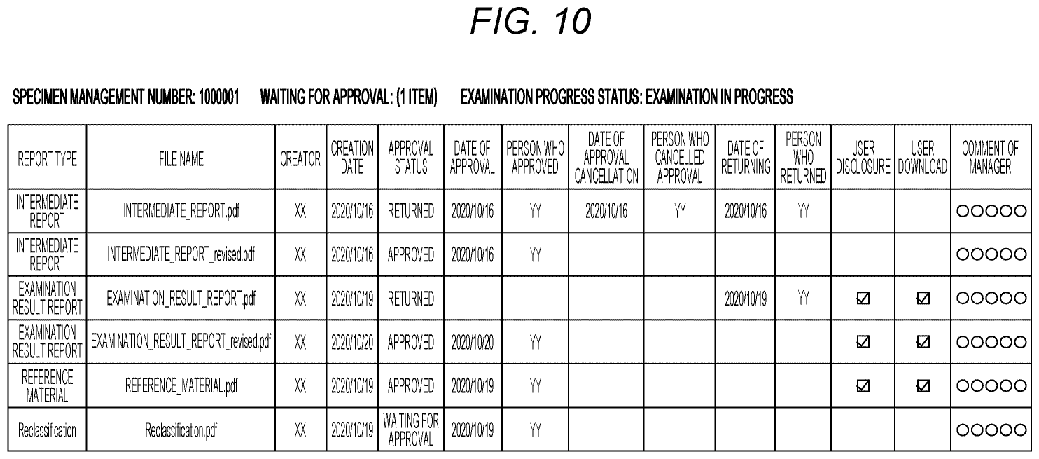

Alternatively, the authorized manager 60 may transmit, after checking and approving the contents, an examination report separately received from the person in charge 50 of the examination, from the transmission/reception unit 130 of the terminal device 100b used by the authorized manager 60 to the transmission/reception unit 230 of the server device and register the examination report in the examination information management system 10. The control unit 210 of the server device 200 permits the terminal device 100b used by the authorized manager 60 to transmit information including the type of examination information, whether the examination report has been approved or returned, and whether or not the examination report has been downloaded by the user in the medical institution. FIG. 10 is an example of information displayed on the display unit 110 of the terminal device 100b used by the authorized manager 60 at this time.

As illustrated in FIG. 10, the display unit 110 of the terminal device 100b used by the authorized manager 60 displays “report type” indicating the type of the examination information, “file name”, “creator” indicating the person in charge 50 of the examination, creation date, “approval status” indicating whether being approved, returned, or in a state of waiting for approval, a date of approval, and whether or not having been disclosed and downloaded to a user in the medical institution. As illustrated in FIG. 10, the authorized manager 60 can cancel approval of the examination result report once approved and return the examination result report to the person in charge 50 of the examination. The information indicating the cancellation of the approval described above and the comment input by the authorized manager 60 are also transmitted from the transmission/reception unit 230 of the server device 200 to the terminal device 100b used by the authorized manager 60, and can be displayed on the display unit 110, under the control of the control unit 210. The authorized manager 60 may simply select examination information to be approved on the basis of this screen and approve the examination information.

Further, the authorized manager 60 can transmit announcement information to another user. Specifically, when the authorized manager 60 inputs announcement information from the operation reception unit 120 of the terminal device 100b used by the authorized manager 60, the announcement information is transmitted from the transmission/reception unit 130 to the server device 200. When the server device 200 receives the announcement information by the transmission/reception unit 230, the server device 200 transmits the announcement information from the transmission/reception unit 230 of the server device 200 to the terminal device 100b used by another user and causes the announcement information to be displayed on the display unit 110, under the control of the control unit 210.

Note that a range of users to which the announcement information is transmitted can be selected from all users, users belonging to a specific belonging destination (for example, a specific medical institution, a specific clinical department, or the like), a responsible person in each medical institution or the like, a specific user, and the like. These pieces of information are also transmitted from the transmission/reception unit 130 to the server device 200 when the information is input from the operation reception unit 120 of the terminal device 100b used by the authorized manager 60. Then, the control unit 210 of the server device 200 controls a transmission destination of the announcement information on the basis of the received information.

Further, a notification destination of the announcement information may be the terminal device 100b used by each user or may be an e-mail address or a telephone number. The control unit 210 of the server device 200 may control these notification destinations on the basis of information transmitted from the terminal device 100b used by the authorized manager 60.

Further, the authorized manager 60 can also refer to account information of another user, and edit, add, and delete the account information. For example, the authorized manager 60 who is also a responsible person in the examination institution can edit, add, and delete account information of other users in the medical institution. When the authorized manager 60 requests the server device 200 to transmit the account information from the terminal device 100 used by the authorized manager 60, the control unit 210 of the server device 200 permits transmission of the account information to the terminal device 100 used by the authorized manager 60. Then, the account information transmitted from the transmission/reception unit 230 of the server device 200 is received by the transmission/reception unit 130 of the terminal device 100b used by the authorized manager 60 and displayed on the display unit 110. When the authorized manager 60 inputs editing, adding, and deleting of the account information from the operation reception unit 120, and the input information is transmitted from the transmission/reception unit 130 of the terminal device 100 used by the authorized manager 60, the control unit 210 of the server device 200 permits reception of the information and change of the account information of the user stored in the storage unit 220.

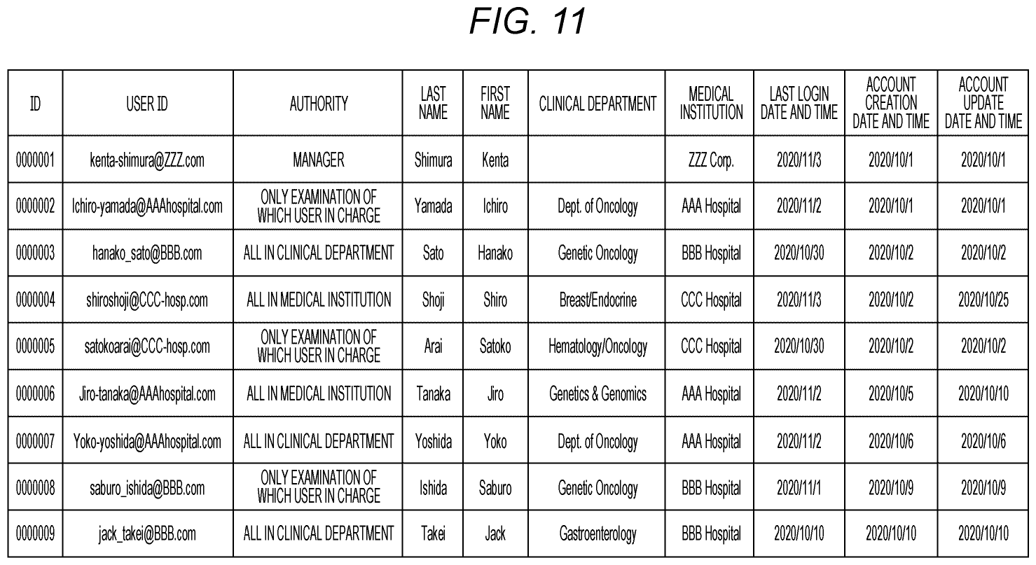

FIG. 11 is an example of information displayed on the display unit 110 of the terminal device 100b used by the authorized manager 60 at this time. In FIG. 11, a list of account information of users is displayed on the display unit 110 of the terminal device 100b used by the authorized manager 60. The displayed account information includes: “ID” which is a unique number indicating a user; “user ID” used when each user logs in to the examination information management system 10 (in the present embodiment, the user ID also serves as an e-mail address for transmitting a notification indicating that new examination information has been registered or announcement notification); “authority” indicating whether or not each user is a manager, a range of accessible examination information, and the like; “last name” and “first name” indicating each user; “clinical department” and “medical institution”, “last login date and time”, and “account creation date and time”, and “account update date and time”. Note that information other than these may be indicated. Further, in FIG. 11, only the date is displayed for the last login date and time, the account creation date and time, and the account update date and time, but information transmitted to the terminal device 100b used by the authorized manager 60 and displayed on the display unit 110 may include more detailed information such as hours, minutes, and seconds. For example, a user with many days having passed after the last login date and time may have already left the job. Therefore, the authorized manager 60 can check an enrollment status of the user and delete the account of the user if the user has left the job.

Alternatively, account information (see FIG. 5) of a specific user may be transmitted to the terminal device 100b used by the authorized manager 60 at this time and displayed on the display unit 110. From the screen, the user's account may be changed, added, and deleted, the manager authority may be granted, a person in charge of the system may be set, the access authority may be changed, and the like.

### OTHER EMBODIMENTS

Note that the embodiment described above is merely an example of implementation in carrying out the present invention, and the technical scope of the present invention should not be interpreted in a limited manner by the embodiment described above. The present invention can be implemented in various forms without departing from the scope or main features of the present invention.

For example, in the embodiment described above, the manager (the authorized manager 60) of the examination institution sets the access authority of individual users, but a manager (another person in charge 40) in a medical institution or a doctor in charge (the person in charge 20 of medical treatment) in a medical institution may set the access authority of a predetermined user.

Further, in the embodiment described above, the examination information management system using the cloud server has been described, but the cloud server is not essential. In addition, a single device (examination information management device) may have a function of a control unit in the examination information management system described above.

### INDUSTRIAL APPLICABILITY

The present invention is useful as an information management system for a medical examination.

### REFERENCE SIGNS LIST

10 Examination information management system
20 Person in charge of medical treatment
30 Counselor in charge
40 Other person in charge
50 Person in charge of examination
60 authorized manager
100 Terminal device
110 Display unit
120 Operation reception unit
130 Transmission/reception unit
200 Server device
210 Control unit
220 Storage unit
230 Transmission/reception unit
NT Network

1. An examination information management system comprising:
a transmitter that transmits, to a user terminal used by a user, examination information regarding an examination performed in association with medical treatment; and
a hardware processor that controls transmission to the user terminal by the transmitter, in accordance with a characteristic that is set for the user and is for indicating a range of the examination information accessible by the user, wherein
by control according to the characteristic, the hardware processor enables transmission of the examination information to the user terminal even when the user is a person other than a person in charge of the medical treatment.

2. The examination information management system according to claim 1, wherein
the hardware processor causes the transmitter to transmit, to the user, the examination information included in a range indicated by the characteristic.

3. The examination information management system according to claim 1, wherein
the characteristic is independently set for a plurality of the users, and
the hardware processor enables the transmission, to the user, of the examination information included in a range indicated by a characteristic set for each of the users.

4. The examination information management system according to claim 1, wherein
a characteristic according to a characteristic indicating an attribute related to a belonging destination of the user is set to the user, and
the hardware processor enables the transmission, to the user, of the examination information included in the set range.

5. The examination information management system according to claim 4, wherein the attribute regarding a belonging destination of the user is any of a medical institution to which the user belongs, a clinical department to which the user belongs, and whether or not the user is a person in charge of the medical treatment.

6. The examination information management system according to claim 1, wherein the transmitter transmits a result report regarding the examination to the user.

7. The examination information management system according to claim 6, wherein the transmitter transmits, to the user, information indicating that the result report has been read or downloaded by a terminal of the user.

8. The examination information management system according to claim 6, wherein
the transmitter transmits, to the user, information indicating that the result report has been registered, and
transmits reminder information to the user when the result report is not read or not downloaded by the user until a predetermined period elapses after transmission of the information indicating the registration.

9. The examination information management system according to claim 1, wherein the transmitter transmits, to the user, information indicating a collection date of a specimen related to the examination or a scheduled completion date of a report.

10. The examination information management system according to claim 1, wherein the transmitter transmits, to the user, information indicating a scheduled completion date of a report, the scheduled completion date being calculated from information indicating a collection date of a specimen related to the examination.

11. The examination information management system according to claim 1, wherein presence or absence of manager authority is set to the user.

12. The examination information management system according to claim 11, wherein the hardware processor permits the user having manager authority to transmit all pieces of the examination information.

13. The examination information management system according to claim 11, comprising:
a receiver that receives examination information transmitted from a user terminal used by a user, wherein
the hardware processor permits reception of the examination information regarding the examination information in the transmission.

14. The examination information management system according to claim 13, wherein
the receiver receives information indicating approval of the examination information and transmitted from the user having manager authority, and
the hardware processor permits transmission, to another user, of only the examination information for which the information indicating the approval is received.

15. The examination information management system according to claim 11, comprising:
a receiver that receives announcement information transmitted from the user having manager authority, wherein
the hardware processor permits transmission of the announcement information to another user.

16. The examination information management system according to claim 11, wherein the hardware processor permits transmission, to the user having manager authority, and reception of information regarding an account of another user.

17. The examination information management system according to claim 11, wherein the hardware processor permits transmission of information regarding a time when another user has performed final login, to the user having manager authority.

18. The examination information management system according to claim 11, wherein
the hardware processor permits transmission, to the user, of information indicating that a result report for the examination has been registered, and
permits transmission, to the user having manager authority, of information indicating whether or not the transmitter has transmitted the result report to the user.

19. An examination information management device for managing transmission of examination information regarding an examination performed in association with medical treatment, the examination information management device comprising:
a hardware processor that controls transmission of the examination information such that the examination information regarding the medical treatment can be transmitted to a user terminal used by the user even when the user is a person other than a person in charge of the medical treatment, in accordance with a characteristic for indicating a range of the examination information accessible by the user.

20. The examination information management system according to claim 2, wherein
the characteristic is independently set for a plurality of the users, and
the hardware processor enables the transmission, to the user, of the examination information included in a range indicated by a characteristic set for each of the users.
